(19) United States
(12) Patent Application Publication
(10) Pub. No.: US 2008/0071349 A1
(43) Pub. Date: Mar. 20, 2008

(54) Medical Devices

(75) Inventors: Liliana Atanasoska, Edina, MN (US); Jan Weber, Maastrich (NL); Steven R. Larsen, Lino Lakes, MN (US); Robert W. Warner, Woodbury, MN (US)

Correspondence Address: FISH & RICHARDSON PC, P.O. BOX 1022, MINNEAPOLIS, MN, 55440-1022, US

(73) Assignee: BOSTON SCIENTIFIC SCIMED, INC., Maple Grove, MN (US)

(21) Appl. No.: 11/854,981

(22) Filed: Sep. 13, 2007

Related U.S. Application Data
(60) Provisional application No. 60/845,298, filed on Sep. 18, 2006.

(51) Int. Cl. A61F 2/06

(52) U.S. Cl. 623/1.15; 623/1.38

(57) ABSTRACT

Medical devices are described that include a device body that carries a first bioerodible member and a second bioerodible member. One of the first or second members includes a bioerodible metallic material or ceramic, and the other includes a bioerodible polymeric material. The first and/or second member can include a therapeutic agent such as paclitaxel.

### CROSS-REFERENCE TO RELATED APPLICATIONS

This application claims priority under 35 USC § 119(e) to U.S. Provisional Patent Application Ser. No. 60/845,298, filed on Sep. 18, 2006, the entire contents of which are hereby incorporated by reference.

### TECHNICAL FIELD

This disclosure relates to medical devices, and to methods of making the same.

### BACKGROUND

The body includes various passageways such as arteries, other blood vessels, and other body lumens. These passageways sometimes become occluded or weakened. For example, the passageways can be occluded by a tumor, restricted by plaque, or weakened by an aneurysm. When this occurs, the passageway can be reopened or reinforced with a medical endoprosthesis. An endoprosthesis is typically a tubular member that is placed in a lumen in the body. Examples of endoprostheses include stents, covered stents, and stent-grafts.

Endoprostheses can be delivered inside the body by a catheter that supports the endoprosthesis in a compacted or reduced-size form as the endoprosthesis is transported to a desired site. Upon reaching the site, the endoprosthesis is expanded, e.g., so that it can contact the walls of the lumen.

The expansion mechanism may include forcing the endoprosthesis to expand radially. For example, the expansion mechanism can include the catheter carrying a balloon, which carries a balloon-expandable endoprosthesis. The balloon can be inflated to deform and to fix the expanded endoprosthesis at a predetermined position in contact with the lumen wall. The balloon can then be deflated, and the catheter withdrawn from the lumen.

### SUMMARY

This disclosure generally relates to medical devices that are, or that include portions that are, erodible or bioerodible. Many of the medical devices disclosed can be configured to deliver therapeutic agents in a controlled and predetermined manner to specific locations of the body for extended periods of time.

In one aspect, the invention features therapeutic agent release assemblies that include a first bioerodible member and a second bioerodible member. One of the first or second members includes a bioerodible metallic material or ceramic and the other includes a bioerodible polymeric material and a therapeutic agent. The first and second members erode in succession.

The release assemblies can further include, e.g., a third, a fourth, a fifth, a sixth, or even a seventh bioerodible member. For example, the release assemblies can further include a third bioerodible member that includes a bioerodible metallic material or ceramic and a fourth bioerodible member that includes a bioerodible polymeric material and the therapeutic agent or a different therapeutic agent.

The therapeutic agent can be a genetic therapeutic agent, a non-genetic therapeutic agent, or cells. Therapeutic agents can be used singularly, or in combination. Therapeutic agents can be, e.g., nonionic, or they may be anionic and/or cationic in nature. A preferred therapeutic agent is one that inhibits restenosis. A specific example of one such therapeutic agent that inhibits restenosis is paclitaxel or derivatives thereof, e.g., docetaxel.

In another aspect, the invention features medical devices that have a device body that carries a first bioerodible member and a second bioerodible member. One of the first or second members includes a bioerodible metallic material or ceramic, and the other includes a bioerodible polymeric material.

The medical device can be, e.g., in the form of an endoprosthesis, e.g., a stent. Other medical devices include stent-grafts and filters.

In embodiments, the first bioerodible member and the second bioerodible member erode in succession. If desired, one or more members can be isolated, at least in part, from the body environment by the device body. For example, one or more members can be carried in a well in the device body.

If desired, the device body can be formed of a non-erodible material. The non-erodible material can be, e.g., a polymeric material, such as polycyclooctene (PCO), styrene-butadiene rubber, polyvinyl acetate, polyvinylidinefluoride (PVDF), polymethylmethacrylate (PMMA), polyurethanes, polyethylene, polyvinyl chloride (PVC), or blends or these materials, or the non-erodible material can be, e.g., a metallic material, such as stainless steel, nitinol, niobium, zirconium, platinum-stainless steel alloy, iridium-stainless steel alloy, titanium-stainless steel alloy, molybdenum, rhenium, or molybdenum-rhenium alloy.

If desired, a therapeutic agent can be disposed within and/or on one or more members.

The medical device can be such that the device body and the first member each include a metallic material, which together define a galvanic couple having a standard cell potential greater than about +0.25 V, e.g., +0.75 V or +1.25 V.

In particular embodiments, the medical device is in the form of an endoprosthesis in which the device body is an endoprosthesis body, and the first and second members are carried in a well defined in the endoprosthesis body.

In another aspect, the invention features methods of making medical devices that include providing a device body having a well and/or an aperture defined therein; providing a first bioerodible member and a second bioerodible member in which one of the first or second members includes a bioerodible metallic material or ceramic and the other includes a bioerodible polymeric material; and placing the first and second members in the well and/or the aperture.

Aspects and/or embodiments may have one or more of the following advantages. Release of a therapeutic agent from a medical devices can be controlled and predetermined. For example, one or more therapeutic agents can be released within a subject sequentially and/or intermittently. Release from the medical device can occur for extended periods of time, e.g., days, months, or even years. If implanted, the medical devices may not need to be removed from the body after implantation. Lumens implanted with such devices can exhibit reduced restenosis. The medical devices can have a low thrombogenecity. Surfaces of such medical devices can support cellular growth (endothelialization), often minimizing the risk of fragmentation as the medical device or portion of the medical devise erodes or bioerodes.

An erodible or bioerodible medical device, e.g., a stent, refers to a device, or a portion thereof, that exhibits substantial mass or density reduction or chemical transformation, after it is introduced into a patient, e.g., a human patient. Mass reduction can occur by, e.g., dissolution of the material that forms the device and/or fragmenting of the device. Chemical transformation can include oxidation/reduction, hydrolysis, substitution, electrochemical reactions, addition reactions, or other chemical reactions of the material from which the device, or a portion thereof, is made. The erosion can be the result of a chemical and/or biological interaction of the device with the body environment, e.g., the body itself or body fluids, into which it is implanted and/or erosion can be triggered by applying a triggering influence, such as a chemical reactant or energy to the device, e.g., to increase a reaction rate. For example, a device, or a portion thereof, can be formed from an active metal, e.g., Mg or Ca or an alloy thereof, and which can erode by reaction with water, producing the corresponding metal oxide and hydrogen gas (a redox reaction). For example, a device, or a portion thereof, can be formed from an erodible or bioerodible polymer, or an alloy or blend erodible or bioerodible polymers which can erode by hydrolysis with water. The erosion occurs to a desirable extent in a time frame that can provide a therapeutic benefit. For example, in embodiments, the device exhibits substantial mass reduction after a period of time which a function of the device, such as support of the lumen wall or drug delivery is no longer needed or desirable. In particular embodiments, the device exhibits a mass reduction of about 10 percent or more, e.g. about 50 percent or more, after a period of implantation of one day or more, e.g. about 60 days or more, about 180 days or more, about 600 days or more, or 1000 days or less. In embodiments, the device exhibits fragmentation by erosion processes. The fragmentation occurs as, e.g., some regions of the device erode more rapidly than other regions. The faster eroding regions become weakened by more quickly eroding through the body of the endoprosthesis and fragment from the slower eroding regions. The faster eroding and slower eroding regions may be random or predefined. For example, faster eroding regions may be predefined by treating the regions to enhance chemical reactivity of the regions. Alternatively, regions may be treated to reduce erosion rates, e.g., by using coatings. In embodiments, only portions of the device exhibits erodibilty. For example, an exterior layer or coating may be erodible, while an interior layer or body is non-erodible. In embodiments, the endoprosthesis is formed from an erodible material dispersed within a non-erodible material such that after erosion, the device has increased porosity by erosion of the erodible material.

Erosion rates can be measured with a test device suspended in a stream of Ringer's solution flowing at a rate of 0.2 m/second. During testing, all surfaces of the test device can be exposed to the stream. For the purposes of this disclosure, Ringer's solution is a solution of recently boiled distilled water containing 8.6 gram sodium chloride, 0.3 gram potassium chloride, and 0.33 gram calcium chloride per liter.

As used herein, metallic material means a pure metal, a metal alloy or a metal composite.

All publications, patent applications, patents, and other references mentioned herein are incorporated by reference herein in their entirety.

Other aspects, features, and advantages will be apparent from the description and drawings, and from the claims.

### DESCRIPTION OF DRAWINGS

FIGS. 1A-1C are longitudinal cross-sectional views, illustrating delivery of a therapeutic agent eluting stent in a collapsed state, expansion of the stent, and the deployment of the stent.

FIG. 2 is a perspective view of the unexpanded therapeutic agent eluting stent of FIG. 1A, illustrating wells defined in a stent body that are each filled with a controlled release assembly.

FIG. 2A is a transverse cross-sectional view of the stent of FIG. 2, taken along 2A-2A.

FIGS. 3A-3D are a sequence of cross-sectional views of the stent of FIG. 2 in a lumen after expansion; FIG. 3A being the stent immediately after implantation in the lumen; FIG. 3B being the stent just after a start of erosion of the assembly; FIG. 3C being the stent after the erosion of the assembly is underway; and FIG. 3D being the stent after erosion of the assembly is complete.

FIG. 3E is a idealized graph showing concentration of a therapeutic agent proximate the release assembly during various states of erosion versus time.

FIG. 4 is a sequence of perspective views illustrating a method of making the stent of FIG. 2.

FIG. 5 is a highly enlarged cross-sectional view of a porous material having interconnected small and large voids.

FIG. 6 is a perspective view of a fenestrated pre-stent prior to insertion of the release assemblies.

FIG. 7 is a perspective view of a wire pre-stent prior to insertion of the release assemblies.

### DETAILED DESCRIPTION

Generally, medical devices are provided that can be configured to deliver therapeutic agents in a controlled and predetermined manner to specific locations in the body for extended periods of time. For example, some devices are configured to release one or more therapeutic agents within a subject, e.g., a mammal, sequentially and/or intermittently.

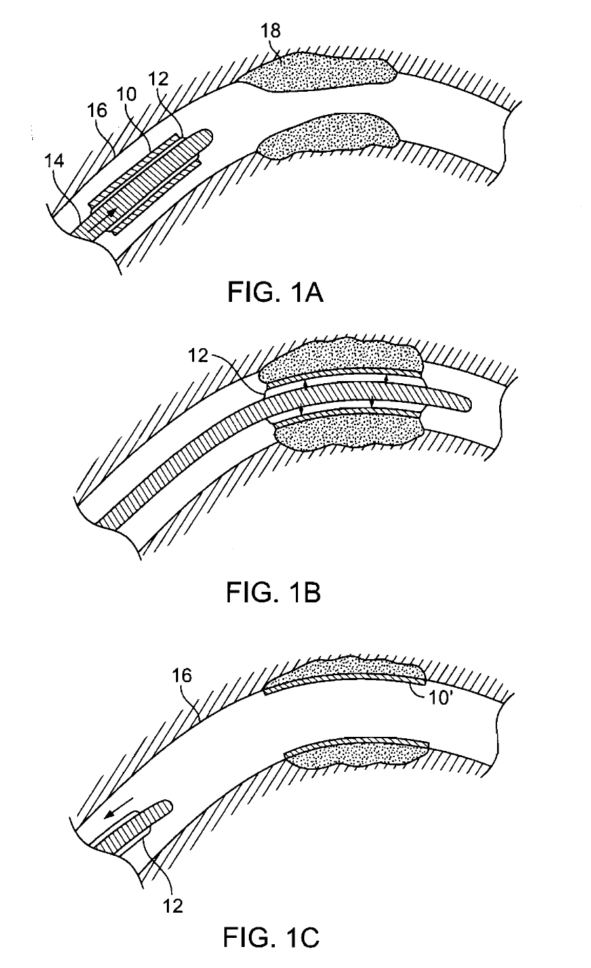

Referring to FIGS. 1A-1C, a therapeutic agent eluting stent 10 is placed over a balloon 12 carried near a distal end of a catheter 14, and is directed through a lumen 16 (FIG. 1A) until the portion carrying the balloon and stent reaches the region of an occlusion 18. The stent is then radially expanded by inflating the balloon 12 and compressed against the vessel wall with the result that occlusion 18 is compressed, and the vessel wall surrounding it undergoes a radial expansion (FIG. 1B). The pressure is then released from the balloon and the catheter is withdrawn from the vessel (FIG. 1C), leaving expanded stent 10′ fixed within lumen 16.

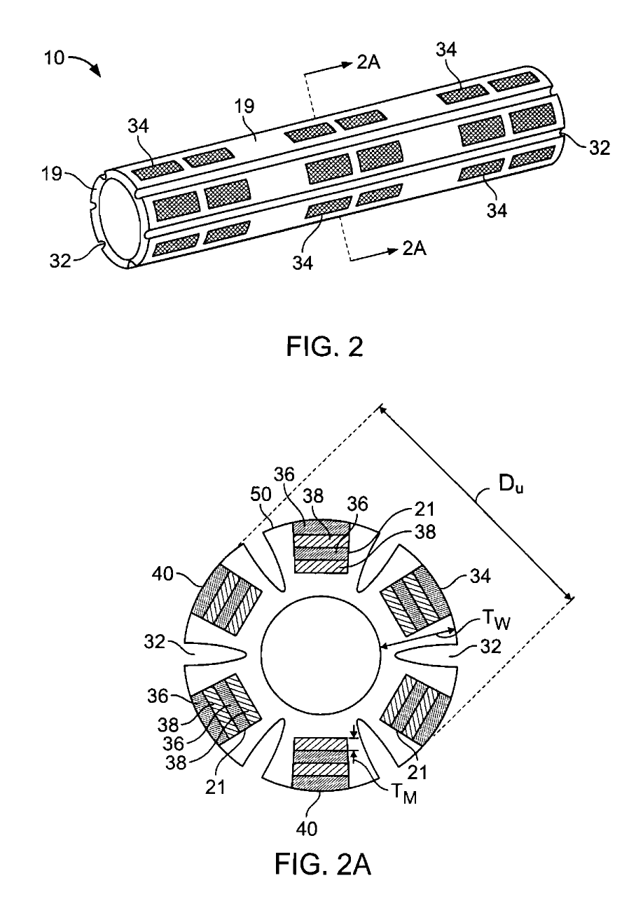

Referring to FIGS. 2 and 2A, unexpanded therapeutic agent eluting stent 10 has a stent body 19, e.g., made of a metallic or a polymeric material, which carries a plurality therapeutic agent release assemblies 34 in wells 21 defined in the stent body 19. In addition to wells 21, stent body 19 defines a plurality of longitudinally extending channels 32 that run an entire longitudinal length of the stent body. In the particular embodiment shown, each release assembly 34 is made up of alternating first 36 and second members 38. Each first member 36 is made of a bioerodible metallic material, e.g., magnesium, or ceramic, e.g., calcium phosphate, and each second member 38 is made of a bioerodible polymeric material, such as polylactic acid or polyglycolic acid. Each second member 38 has a therapeutic agent such as paclitaxel (taxol) dispersed therein. As illustrated, each first 36 and second member 38 is dimensionally similar and have substantially planar sides, except that each outermost first member 40 that will contact a lumen wall when expanded, is radiused to match the radius of curvature of the stent body 19. As such, each outermost first member 40 forms part of a generally smooth outer wall 50. In addition, each member of each assembly, and each assembly itself, is sized to fit into each well 21 with a substantially water-tight fit such outer members substantially protect and isolate inner members from the body environment. Such a stent configuration allows for intermittent delivery of one or more therapeutic agents to a specific location of the body of a subject over extended periods of time, as will be described in further detail below.

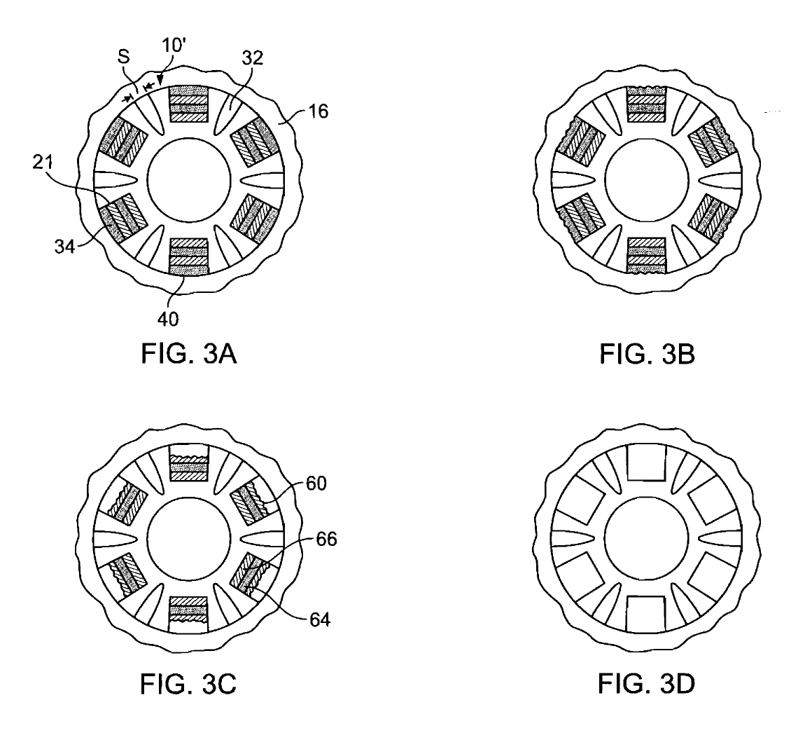

Referring also now to FIGS. 3A-3D, during expansion, stent 10 preferentially expands along channels 32 because stent body 19 is thinnest at the bottom of the channels, opening up the circumferential spacing S between opposite channel boundaries along the outer surface the stent. This expansion mode leaves dimensions of wells 21 substantially unchanged, maintaining the water-tight fit of each assembly 34 in each well 21. Immediately following insertion into lumen 16, each assembly 34 of the expanded stent 10′ is in a substantially non-eroded state (FIG. 3A). However, once inserted, body fluids and substances in the body fluids begin to attack, e.g., chemically attack, the outermost first members 40 (FIG. 3B), while the outermost first members 40 substantially protect and isolate inner members from the body environment. For example, when outermost first member is magnesium, water begins to react with the magnesium metal, producing hydrogen gas and magnesium hydroxide. No therapeutic agent is released during the period of erosion of the outermost first members since these member do not include a therapeutic agent, and those members that do include a therapeutic agent are protected from the body environment until the outermost first members have completely eroded. After each outermost first member has completely eroded, the outermost second members 60 that are each made of a bioerodible polymeric having a therapeutic agent dispersed therein begin to erode (FIG. 3C) with the release of therapeutic agent. After outermost second members have completely eroded, the innermost first members 64 that are made of a bioerodible metallic material or ceramic begin to erode. Again, no therapeutic agent is released during this period because these members do not include a therapeutic agent. After innermost first member has completely eroded, the innermost second members 66 that are each made of a bioerodible polymeric having a therapeutic agent dispersed therein begin to erode with the release of therapeutic agent. After the innermost second members completely erode, therapeutic agent release stops (FIG. 3D).

Referring now also to FIG. 3E, at least one of the results of the sequential erosion just described is intermittent release of the therapeutic agent or agents from the stent. While FIG. 3E is an idealized concentration versus time graph and other concentration versus time profiles are possible, it does illustrate that during erosion of first members 40 and 64, no therapeutic agent is released, resulting in a concentration proximate the release assemblies that is substantially zero. It also illustrates that when the second members 60 and 66 are eroding, there is release of therapeutic agent. As shown, at least in some embodiments, release has an idealized “zero order” profile (constant concentration over the time period). Other release profiles are possible. Lumens implanted with such release assemblies can exhibit reduced restenosis over the long term because a therapeutic agent can be released more than once after implantation of the stent.

Generally, the unexpanded diameter Dᵤ (FIG. 2A) and the unexpanded wall thickness T_{W} of stent 10 will depend upon the strength required for the desired application of the stent and the material from which the stent body 19 is formed. In embodiments, the unexpanded diameter Dᵤ is between about 3 mm and about 15 mm, e.g., between about 4 mm and about 10 mm. In embodiments, the wall thickness T_{W} is between about 1.0 mm and about 7 mm, e.g., between about 1.5 mm and about 5 mm. Generally when the device body is formed from a polymeric material, larger wall thicknesses are desirable in comparison to a device body formed from a metallic material or a ceramic.

Generally, first and second members have a thickness T_{M} (FIG. 2A) and cross-sectional area that consistent with desired degradation and therapeutic agent release rate, and the desired application. Thickness and cross-sectional area of the members can be used to control release rate and timing of the release. In embodiments, the thickness of the members is from about 0.25 mm to about 1.5 mm, e.g., between about 0.5 mm and about 1.0 mm. In embodiments, each first and second members have a cross-sectional area of 0.1 mm² to about 1 mm², e.g., from about 0.25 mm² to about 0.75 mm².

First and second members can be made, e.g., by extrusion, molding or casting. If desired, the members can be machined to size, e.g. using Computer Numerical Control (CNC).

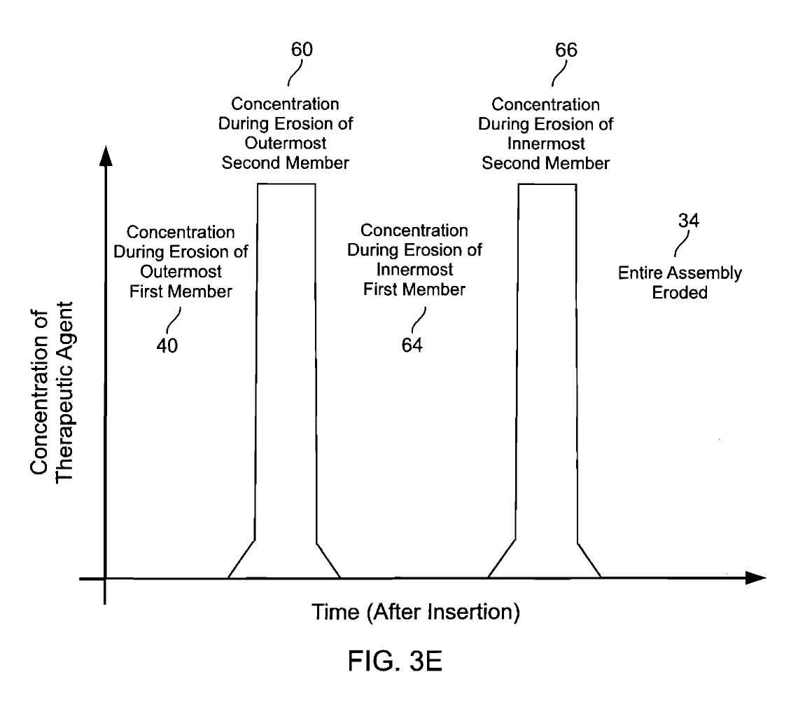

Referring now to FIG. 4, stent 10 of FIG. 2 can be made by providing a pre-device body 19′ having channels defined therein. Such a pre-device body 19′ can be made, e.g., by profile extrusion. Wells 21 are then formed in pre-device body 19′, e.g., using CNC laser ablation, to form device body 19. Unexpanded stent 10 is then completed by placing first and second members into wells 21 in the desired sequence, e.g., using a pick-and-place robot. Robots capable of assembling very small parts are available from EPSON (E2 Robots) and Yamaha (e.g., YK180X or YK220X). Individual members can be friction fit into wells 21, optionally, using an adhesive to help secure them in place, or the members can first be assembled outside the wells in the desired order, e.g., by using a bioerodible adhesive, and then each assembly can be press fit into wells 21.

The stent body can be made from one or more bioerodible metals or a metal alloys. Examples of bioerodible metals include iron, magnesium, zinc, aluminum and calcium. Examples of metallic alloys include iron alloys having, by weight, 88-99.8% iron, 0.1-7% chromium, 0-3.5% nickel, and less than 5% of other elements (e.g., magnesium and/or zinc); or 90-96% iron, 3-6% chromium and 0-3% nickel plus 0-5% other metals. Other examples of alloys include magnesium alloys, such as, by weight, 50-98% magnesium, 0-40% lithium, 0-5% iron and less than 5% other metals or rare earths; or 79-97% magnesium, 2-5% aluminum, 0-12% lithium and 1-4% rare earths (such as cerium, lanthanum, neodymium and/or praseodymium); or 85-91% magnesium, 6-12% lithium, 2% aluminum and 1% rare earths; or 86-97% magnesium, 0-8% lithium, 2%-4% aluminum and 1-2% rare earths; or 8.5-9.5% aluminum, 0.15%-0.4% manganese, 0.45-0.9% zinc and the remainder magnesium; or 4.5-5.3% aluminum, 0.28%-0.5% manganese and the remainder magnesium; or 55-65% magnesium, 30-40% lithium and 0-5% other metals and/or rare earths. Magnesium alloys are available under the names AZ91D, AM50A, and AE42, which are available from Magnesium-Elektron Corporation (United Kingdom). Still other magnesium alloys include AZ, AS, ZK, AM, LAE, WE alloys and others discussed in Aghion et al., JOM, page 30 (November 2003), and Witte et al., Biomaterials, 27, 1013-1018 (2006). Other erodible metals or metal alloys are described in Bolz, U.S. Pat. No. 6,287,332 (e.g., zinc-titanium alloy and sodium-magnesium alloys); Heublein, U.S. Patent Application 2002/0004060; Kaese, Published U.S. Patent Application No. 2003/0221307; Stroganov, U.S. Pat. No. 3,687,135; and Park, Science and Technology of Advanced Materials, 2, 73-78 (2001).

The stent body can be made from one or more bioerodible ceramics. Examples of bioerodible ceramics include beta-tertiary calcium phosphate (β-TCP), blends of β-TCP and hydroxy apatite, CaHPO₄, CaHPO₄-2H₂O, CaCO₃ and CaMg(CO₃)₂. Other bioerodible ceramics are discussed in Zimmermann, U.S. Pat. No. 6,908,506, and Lee, U.S. Pat. No. 6,953,594.

The stent body can be made from one or more bioerodible polymers. Examples of bioerodible polymers include polycaprolactone (PCL), polycaprolactone-polylactide copolymer (e.g., polycaprolactone-polylactide random copolymer), polycaprolactone-polyglycolide copolymer (e.g., polycaprolactone-polyglycolide random copolymer), polycaprolactone-polylactide-polyglycolide copolymer (e.g., polycaprolactone-polylactide-polyglycolide random copolymer), polylactide, polycaprolactone-poly(β-hydroxybutyric acid) copolymer (e.g., polycaprolactone-poly(β-hydroxybutyric acid) random copolymer) poly(β-hydroxybutyric acid), polyvinyl alcohol, polyethylene glycol, polyanhydrides and polyiminocarbonates, and mixtures of these polymers. Additional examples of bioerodible polymers are described in Sahatjian et. al, U.S. Published Patent Application No. 2005/0251249.

The stent body can be made of one or more non-erodible metals or metal alloys. Examples of non-erodible metals and metal alloys include stainless steel, nitinol, niobium, zirconium, platinum-stainless steel alloy, iridium-stainless steel alloy, titanium-stainless steel alloy, molybdenum, rhenium, molybdenum-rhenium alloys, cobalt-chromium, and nickel, cobalt, chromium, molybdenum alloy (e.g., MP35N).

The stent body can be made from one or more non-bioerodible polymers. Examples of non-bioerodible polymers include polycyclooctene (PCO), styrene-butadiene rubber, polyvinyl acetate, polyvinylidinefluoride (PVDF), polymethylmethacrylate (PMMA), polyurethanes, polyethylene, polyvinyl chloride (PVC), and blends thereof. Additional examples of non-bioerodible polymers are described in Sahatjian et. al, U.S. Published Patent Application No. 2005/0251249.

The members can be made from one or more bioerodible metals or a metal alloys. Examples of bioerodible metals include iron, magnesium, zinc, aluminum, calcium and any of the other bioerodible metals or a metal alloys discussed above.

The members can be made from one or more bioerodible ceramics. Examples of bioerodible ceramics include beta-tertiary calcium phosphate (β-TCP), blends of β-TCP and hydroxy apatite and any of the other bioerodible ceramics discussed above.

The members can be made from one or more bioerodible polymers. Examples of bioerodible polymers include polycaprolactone (PCL), polycaprolactone-polylactide copolymer (e.g., polycaprolactone-polylactide random copolymer), polycaprolactone-polyglycolide copolymer (e.g., polycaprolactone-polyglycolide random copolymer), polycaprolactone-polylactide-polyglycolide copolymer (e.g., polycaprolactone-polylactide-polyglycolide random copolymer), polylactide and any of the other bioerodible polymers discussed above.

Any of the metallic materials, ceramics or polymeric materials described herein can be made porous.

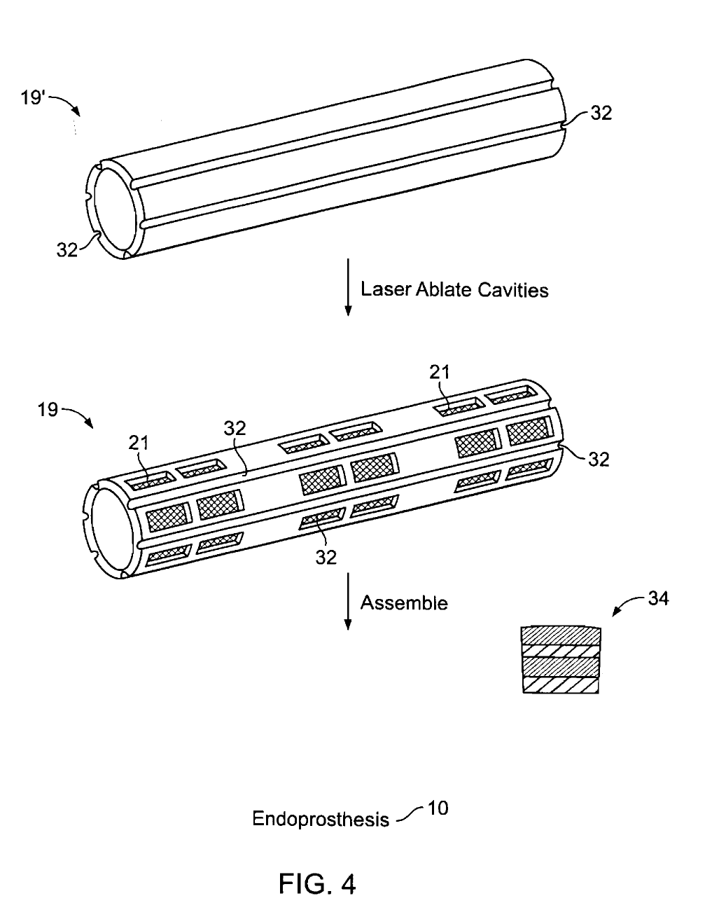

For example, and by reference to FIG. 5, porous metal components can be made by sintering metal particles, e.g., having diameters between about 0.01 micron and 20 micron, to form a porous material 62 having small 63 (e.g., from about 0.05 to about 0.5 micron) and large 65 (e.g., from about 1 micron to about 10 micron) interconnected voids though which a fluid may flow. The microstructure of the porous material can be controlled, e.g., by controlling the particle size and material used, and by controlling the pressure and temperature applied during the sintering process. The voids in the porous material can be, e.g., used as depositories for a therapeutic agent that has been intercalated into the porous material.

For example, such porous materials can have a total porosity, as measured using mercury porosimetry, of from about 80 to about 99 percent, e.g., from about 80 to about 95 percent or from about 85 to about 92 percent, and a specific surface area, as measured using BET (Brunauer, Emmet and Teller), of from about 200 cm²/cm³ to about 10,000 cm²/cm³, e.g., from about 250 cm²/cm³ to about 5,000 cm²/cm³ or from about 400 cm²/cm³ to about 1,000 cm²/cm³. When bioerodible materials are utilized, the porous nature of the material can aid in the erosion of the material, as least in part, due to its increased surface area. In addition, when bioerodible materials are utilized, the porosity of the materials can ensure small fragment sizes. Porous materials and methods of making porous materials is described by Date et al., U.S. Pat. No. 6,964,817; Hoshino et al., U.S. Pat. No. 6,117,592; and Sterzel et al., U.S. Pat. No. 5,976,454.

In some embodiments, the stent body is formed from a bioerodible metal; each first member is formed of a different and electrochemically disparate bioerodible metal, e.g., having a substantially different standard reduction potential than the metal of the stent body; and each second member is formed of bioerodible polymeric material such as polylactic acid having, e.g., a soluble paclitaxel derivative dispersed therein. Furthermore, in such embodiments, each first member is in electrical communication with the stent body, which sets up a galvanic reaction between the disparate metals. For example, a standard cell potential for the galvanic couple can be greater than 2.00 V, e.g., greater than 1.75 V, 1.50 V, 1.00 V, 0.75 V, 0.5 V, 0.35 V, 0.25 V, or greater than 0.15 V. In such instances, one of the metals enhances the erosion of the other metal; while, at the same time, the one of the metals is protected from erosion by the other metal. Galvanic corrosion of a zinc/steel couple is discussed in Tada et al., Electrochimica Acta, 49, 1019-1026 (2004).

Generally, the standard cell potential for a galvanic couple and a ratio of the cathodic-to-anodic area determines the rate of galvanic erosion. A relatively large cathodic-to-anodic area enhances the rate of erosion, while a relatively small cathodic-to-anodic reduces the rate of erosion.

For example, in a particular embodiment, the stent body is formed of iron and each first member is formed of magnesium in electrical communication with the iron stent body. In this instance, the erosion of magnesium is enhanced by the iron; while, at the same time, the erosion of iron is suppressed. For this magnesium-iron couple E^{o}_{Mg—Fe} of 1.94 V. Such a stent configuration can reduce overall degradation time of the entire stent and/or reduce the time between intermittent periods of the release of therapeutic agent. Erosion of magnesium and magnesium alloys is reviewed by Ferrando, J. Mater. Eng., 11, 299 (1989).

In embodiments, the cathode-to-anode ratio is greater than 1. For example, the cathode-to-anode ratio can be greater than 2, 3, 5, 7, 10, 12, 15, 20, 25, 35, or even 50.

In some embodiments, the stent body is formed of a porous bioerodible metal; each first member is formed of a different and electrochemically disparate bioerodible metal; and each second member is formed of bioerodible polymeric material such as polylactic acid having, e.g., a therapeutic agent dispersed therein. Furthermore, in such embodiments, each first member is in electrical communication with the stent body, which sets up a galvanic reaction between the disparate metals. The stent body can be, e.g., intercalated with a therapeutic agent or an erosion-enhancing agent. Erosion-enhancing agents can, e.g., help to oxidize the metallic material and include porphyrins and polyoxymetalates. Porphyrins complexes are described by Suslick et al., New. J. Chem., 16, 633 (1992) and polyxoymetalates are described by Pinnavaia et al., U.S. Pat. No. 5,079,203. Other redox active catalysts are described in Wang, Journal of Power Sources, 152, 1-15 (2005).

In general, the therapeutic agent can be a genetic therapeutic agent, a non-genetic therapeutic agent, or cells. Therapeutic agents can be used singularly, or in combination. Therapeutic agents can be, for example, nonionic, or they may be anionic and/or cationic in nature. A preferred therapeutic agent is one that inhibits restenosis. A specific example of one such therapeutic agent that inhibits restenosis is paclitaxel or derivatives thereof, e.g., docetaxel. Soluble paclitaxel derivatives can be made by tethering solubilizing moieties off the 2′ hydroxyl group of paclitaxel, such as —COCH₂CH₂CONHCH₂CH₂(OCH₂)ₙOCH₃ (n being, e.g., 1 to about 100 or more).

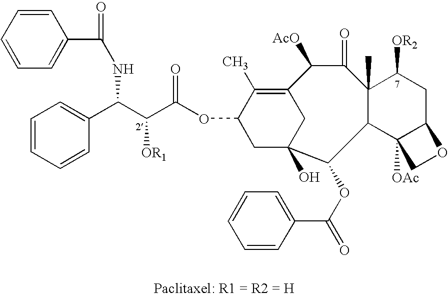

Li et al., U.S. Pat. No. 6,730,699 describes additional water soluble derivatives of paclitaxel.

Exemplary non-genetic therapeutic agents include: (a) anti-thrombotic agents such as heparin, heparin derivatives, urokinase, PPack (dextrophenylalanine proline arginine chloromethylketone), and tyrosine; (b) anti-inflammatory agents, including non-steroidal anti-inflammatory agents (NSAID), such as dexamethasone, prednisolone, corticosterone, budesonide, estrogen, sulfasalazine and mesalamine; (c) anti-neoplastic/antiproliferative/anti-miotic agents such as paclitaxel, 5-fluorouracil, cisplatin, vinblastine, vincristine, epothilones, endostatin, angiostatin, angiopeptin, rapamycin (sirolimus), biolimus, tacrolimus, everolimus, monoclonal antibodies capable of blocking smooth muscle cell proliferation, and thymidine kinase inhibitors; (d) anesthetic agents such as lidocaine, bupivacaine and ropivacaine; (e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides; (i) vascular cell growth promoters such as growth factors, transcriptional activators, and translational promotors; (g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; (h) protein kinase and tyrosine kinase inhibitors (e.g., tyrphostins, genistein, quinoxalines); (i) prostacyclin analogs; (j) cholesterol-lowering agents; (k) angiopoietins; (l) antimicrobial agents such as triclosan, cephalosporins, aminoglycosides and nitrofurantoin; (m) cytotoxic agents, cytostatic agents and cell proliferation affectors; (n) vasodilating agents; (o) agents that interfere with endogenous vasoactive mechanisms; (p) inhibitors of leukocyte recruitment, such as monoclonal antibodies; (q) cytokines, (r) hormones; and (s) antispasmodic agents, such as alibendol, ambucetamide, aminopromazine, apoatropine, bevonium methyl sulfate, bietamiverine, butaverine, butropium bromide, n-butylscopolammonium bromide, caroverine, cimetropium bromide, cinnamedrine, clebopride, coniine hydrobromide, confine hydrochloride, cyclonium iodide, difemerine, diisopromine, dioxaphetyl butyrate, diponium bromide, drofenine, emepronium bromide, ethaverine, feclemine, fenalamide, fenoverine, fenpiprane, fenpiverinium bromide, fentonium bromide, flavoxate, flopropione, gluconic acid, guaiactamine, hydramitrazine, hymecromone, leiopyrrole, mebeverine, moxaverine, nafiverine, octamylamine, octaverine, oxybutynin chloride, pentapiperide, phenamacide hydrochloride, phloroglucinol, pinaverium bromide, piperilate, pipoxolan hydrochloride, pramiverin, prifinium bromide, properidine, propivane, propyromazine, prozapine, racefemine, rociverine, spasmolytol, stilonium iodide, sultroponium, tiemonium iodide, tiquizium bromide, tiropramide, trepibutone, tricromyl, trifolium, trimebutine, tropenzile, trospium chloride, xenytropium bromide, ketorolac, and pharmaceutically acceptable salts thereof.

Exemplary genetic therapeutic agents include anti-sense DNA and RNA as well as DNA coding for: (a) anti-sense RNA, (b) tRNA or rRNA to replace defective or deficient endogenous molecules, (c) angiogenic factors including growth factors such as acidic and basic fibroblast growth factors, vascular endothelial growth factor, epidermal growth factor, transforming growth factor α and β, platelet-derived endothelial growth factor, platelet-derived growth factor, tumor necrosis factor α, hepatocyte growth factor and insulin-like growth factor, (d) cell cycle inhibitors including CD inhibitors, and (e) thymidine kinase (“TK”) and other agents useful for interfering with cell proliferation. Also of interest is DNA encoding for the family of bone morphogenic proteins (“BMP's”), including BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are any of BMP-2, BMP-3, BMP-4, BMP-5, BMP-6 and BMP-7. These dimeric proteins can be provided as homodimers, heterodimers, or combinations thereof, alone or together with other molecules. Alternatively, or in addition, molecules capable of inducing an upstream or downstream effect of a BMP can be provided. Such molecules include any of the “hedgehog” proteins, or the DNA's encoding them.

Vectors for delivery of genetic therapeutic agents include viral vectors such as adenoviruses, gutted adenoviruses, adeno-associated virus, retroviruses, alpha virus (Semliki Forest, Sindbis, etc.), lentiviruses, herpes simplex virus, replication competent viruses (e.g., ONYX-015) and hybrid vectors; and non-viral vectors such as artificial chromosomes and mini-chromosomes, plasmid DNA vectors (e.g., pCOR), cationic polymers (e.g., polyethyleneimine, polyethyleneimine (PEI)), graft copolymers (e.g., polyether-PET and polyethylene oxide-PEI), neutral polymers PVP, SP1017 (SUPRATEK), lipids such as cationic lipids, liposomes, lipoplexes, nanoparticles, or micro particles, with and without targeting sequences such as the protein transduction domain (PTD).

Cells for use include cells of human origin (autologous or allogeneic), including whole bone marrow, bone marrow derived mono-nuclear cells, progenitor cells (e.g., endothelial progenitor cells), stem cells (e.g., mesenchymal, hematopoietic, neuronal), pluripotent stem cells, fibroblasts, myoblasts, satellite cells, pericytes, cardiomyocytes, skeletal myocytes or macrophage, or from an animal, bacterial or fungal source (xenogeneic), which can be genetically engineered, if desired, to deliver proteins of interest.

The therapeutic agent or agents can be carried by one or more members or the stent body. For example, the therapeutic agent can be dispersed within the bioerodible material from which the member and/or device body is formed, or it can be dispersed within an outer layer of the member, such as a coating that forms part of the member and/or stent body.

The stents described herein can be delivered to a desired site in the body by a number of catheter delivery systems, such as a balloon catheter system, as described above. Exemplary catheter systems are described in U.S. Pat. Nos. 5,195,969, 5,270,086, and 6,726,712. The Radius® and Symbiott systems, available from Boston Scientific Scimed, Maple Grove, Minn., also exemplify catheter delivery systems.

The stents described herein can be configured for vascular or non-vascular lumens. For example, they can be configured for use in the esophagus or the prostate. Other lumens include biliary lumens, hepatic lumens, pancreatic lumens, uretheral lumens and ureteral lumens.

Any stent described herein can be dyed or rendered radio-opaque by addition of, e.g., radio-opaque materials such as barium sulfate, platinum or gold, or by coating with a radio-opaque material.

### OTHER EMBODIMENTS

A number of embodiments have been described. Still other embodiments are possible.

For example, while embodiments have been described in which a metal is the outermost member, in some embodiments, a bioerodible polymeric material is the outermost member. This can be advantageous when it is desirable to immediately deliver a therapeutic agent to a lumen, followed by no release, followed by delivery again.

While embodiments have been described in which only two different materials are used in the members of the release assembly, in some embodiments, three, four or even five different materials are employed. Each one of the members can have the same or different therapeutic agent on and/or dispersed therein.

Any member, stent body and/or stent can be coated with a polymeric coating, e.g., a therapeutic agent eluting polymeric coating. This can, e.g., delay or enhance therapeutic agent delivery.

While members have been described that are rectangular in cross-section, other shapes are possible. For example, square, hexagonal or octagonal shapes are possible. In addition, while rectangular shapes are described that do not extend along an entire longitudinal length of the stent body, in some implementations, the rectangular shapes are elongated so that the members extend along the entire longitudinal length of the stent body.

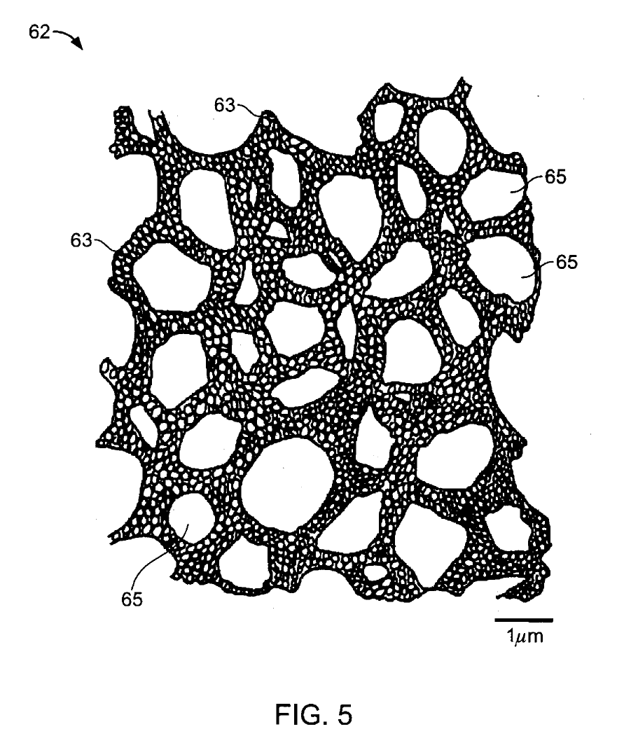

Release assemblies can be placed into apertures, rather than wells. Referring to FIG. 6, a stent body 100 can define a plurality of apertures into which sized release assemblies can be placed. In such embodiments, a therapeutic agent can be delivered to not only a lumen in contact with the stent, but also to any fluid that flows through the stent.

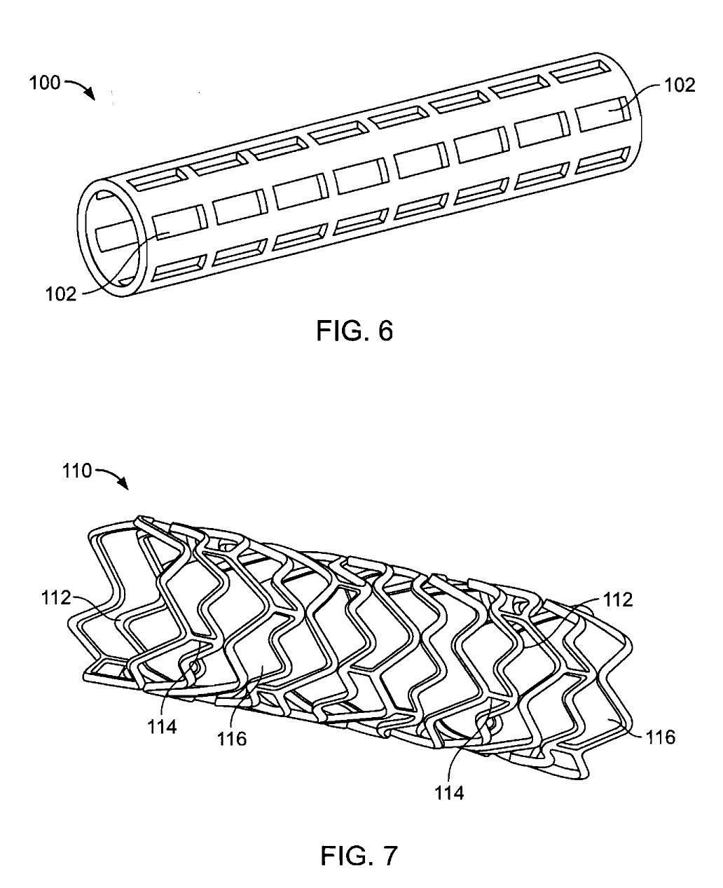

Other stent body forms are possible. For example, a stent body can be in the form of a coil or a wire mesh. Referring to FIG. 7, a wire mesh stent body 110 includes wires 112 and connectors 114 connecting adjacent wires. The wire mesh stent body 110 defines a plurality of openings 116 into which sized release assemblies can be inserted.

Any device body and/or any member can be formed from a bioerodible composite material, such as a composite that includes a polymeric material and metallic material. For example, the body and/or any member can be formed of a composite that includes polylactic acid and iron particles. If desired the composite can include a therapeutic agent and/or and erosion-enhancing agent, such as a metallo-porphyrin.

Medical devices other than stents can be used. For example, therapeutic agent release assemblies can be carried on grafts or filters.

Still other embodiments are within the scope of the following claims.

What is claimed is:

1. A medical device having a device body carrying a first bioerodible member and a second bioerodible member, wherein one of the first or second members comprises a bioerodible metallic material or ceramic, and the other comprises a bioerodible polymeric material.

2. The medical device of claim 1, wherein the first bioerodible member and the second bioerodible member erode in succession.

3. The medical device of claim 2, wherein the first member erodes before the second member.

4. The medical device of claim 2, wherein the second member is substantially isolated from the body environment during erosion of the first member.

5. The medical device of claim 4, wherein erosion of the first member exposes the second member to the body environment.

6. The medical device of claim 2, wherein the second member is isolated, at least in part, from the body environment by the device body.

7. The medical device of claim 2, wherein the first and/or second member is carried in a well in the device body.

8. The medical device of claim 2, wherein the first member is a bioerodible metal and the second member is a bioerodible polymer.

9. The medical device of claim 1, wherein the medical device is in the form of an endoprosthesis.

10. The medical device of claim 1, wherein the device body is formed of a non-erodible material.

11. The medical device of claim 10, wherein the non-erodible material is a polymeric material.

12. The medical device of claim 11, wherein the polymeric material is selected from the group consisting of polycyclooctene (PCO), styrene-butadiene rubber, polyvinyl acetate, polyvinylidinefluoride (PVDF), polymethylmethacrylate (PMMA), polyurethanes, polyethylene, polyvinyl chloride (PVC), and blends thereof.

13. The medical device of claim 10, wherein the non-erodible material is a metallic material.

14. The medical device of claim 13, wherein the metallic material is selected from the group consisting of stainless steel, nitinol, niobium, zirconium, platinum-stainless steel alloy, iridium-stainless steel alloy, titanium-stainless steel alloy, molybdenum, rhenium, and molybdenum-rhenium alloy.

15. The medical device of claim 1, wherein a therapeutic agent is disposed within and/or on the first and/or second member.

16. The medical device of claim 15, wherein the therapeutic agent comprises paclitaxel, or a derivative thereof.

17. The medical device of claim 1, wherein the medical device is in the form of an endoprosthesis in which the device body is an endoprosthesis body, and wherein the first and second members are carried in a well defined in the endoprosthesis body.

18. The medical device of claim 17, wherein the first member comprises a metallic material, and the second member includes a therapeutic agent dispersed within the bioerodible polymeric material.

19. The medical device of claim 17, wherein the first member is carried by the endoprosthesis body such that it defines a portion of an outer surface of the endoprosthesis which is configured to contact a lumen wall.

20. The medical device of claim 19, wherein the second member is in contact with the first member, and is disposed inwardly of the first member.

21. The medical device of claim 20, further comprising a third bioerodible member comprising a bioerodible metallic material or ceramic in contact with the second bioerodible member, and disposed inwardly of the second bioerodible member.

22. The medical device of claim 21, further comprising a fourth bioerodible member comprising a bioerodible polymeric material in contact with the third bioerodible member, and disposed inwardly of the third bioerodible member.

23. The medical device of claim 1, wherein the device body and the first member each comprise a metallic material, which together define a couple, and wherein a standard cell potential for the couple is at least +0.25 V.

24. The medical device of claim 23, wherein the standard cell potential for the couple is at least +0.75 V.

25. A method of making a medical device, the method comprising:
providing a device body having a well and/or an aperture defined therein;
providing a first bioerodible member and a second bioerodible member, wherein one of the first or second members comprises a bioerodible metallic material or ceramic and the other comprises a bioerodible polymeric material; and
placing the first and second members in the well and/or the aperture.

26. A therapeutic agent release assembly comprising a first bioerodible member and a second bioerodible member, wherein one of the first or second members comprises a bioerodible metallic material or ceramic and the other comprises a bioerodible polymeric material and a therapeutic agent, and wherein the first and second members erode in succession.

27. The release assembly of claim 26, further comprising a third bioerodible member comprising a bioerodible metallic material or ceramic.

28. The release assembly of claim 27, further comprising a fourth bioerodible member comprising a bioerodible polymeric material and the therapeutic agent or a different therapeutic agent.
